(19) United States
(12) Patent Application Publication
(10) Pub. No.: US 2003/0087420 A1
(43) Pub. Date: May 8, 2003

(54) Transgenic animals and cells expressing proteins necessary for susceptibility to HIV infection

(75) Inventors: Takashi Yoshiki, Hokkaido (JP); Yorong Lai, Hokkaido (JP); Hitoshi Ikeda, Hokkaido (JP)

Correspondence Address: FOLEY & LARDNER, P.O. Box 80278, San Diego, CA, 92138-0278, US

(73) Assignee: GeneticLab Co., Ltd., Hokkaido (JP)

(21) Appl. No.: 10/176,966

(22) Filed: Jun. 21, 2002

(30) Foreign Application Priority Data
Jun. 25, 2001 (JP) 2001-191416

(51) Int. Cl. C12N 7/00; C12N 5/06; C12N 15/867

(52) U.S. Cl. 435/235.1; 435/353; 435/354; 435/456

(57) ABSTRACT

The present invention provides mammalian cells and mammalian animals that produce HIV particles. The rodent animals of the present invention are able to stably express a human CD4, a human chemokine receptor (such as CXCR4 or CCR5), a human cyclin T1, and a human class II transactivator (CIITA), and produce HIV virus particles. Also provided are methods of preparing the transgenic cells and rodent animals of the invention, as well as methods of using them to identify and assay test agents for anti-HIV activity. Also provided are methods and pharmaceutical compositions for treating and preventing HIV infection in a mammal.

This application claims priority to Japanese Patent Application No. 2001-191416, filed Jun. 25, 2001, which is hereby incorporated by reference in its entirety.

### FIELD OF THE INVENTION

The present invention relates to transgenic animals and animal cells expressing human genes that can be infected by the HIV virus or used to produce HIV particles.

### BACKGROUND OF THE INVENTION

A major obstacle in the study of acquired immunodeficiency syndrome (AIDS) has been the lack of suitable animal models for testing drugs, vaccines, and other agents directed against the HIV-1 virus. Development of animal models has been a serious challenge because the human immunodeficiency virus (HIV) infects human cells through receptors located on the cells that are specific to both the species and the cell type infected. The entry of HIV-1 into a cell requires the presence of human CD4 (hCD4) and a chemokine receptor such as hCXCR4 or hCCR5. (Cormier et al., “An overview of HIV-1 co-receptor function and its inhibitors,” HIV Molecular Immunology 2000 (December 2000)). A viral protein called Tat recruits the human positive transcription elongation factor (p-TEFb) through an RNA stem-loop structure known as the transactivation response (TAR) element, which forms at the 5′ end of the nascent viral transcripts. Cyclin T1, which forms a p-TEFb complex together with cyclin dependent kinase 9 (CDK9), binds to the activation domain of Tat (P. Wei et al., “A novel CDK9-associated C-type cyclin interacts directly with HIV-1 Tat and mediates its high-affinity, loop specific binding to TAR RNA,” Cell 92, 451-462 (1998)). TAR, Tat and Cyclin T1 then form a high affinity RNA-protein complex that phosphorylates the C-terminal domain of RNA-polymerase II (C. Fong et al., “Specific interaction of Tat with the human but not rodent p-TEFb complex mediates the species-specific Tat activation of the HIV-1 transcription,” Proc. Natl. Acad. Sci. USA 96, 2728-2733 (1999); K. Fujinaga et al., “Interactions between human cyclinT, Tat, and the transactivation response element (TAR) are disrupted by a cysteines to tyrosine substitution found in mouse cyclin T,” Proc. Natl. Acad. Sci. USA 96, 1285-1290 (1999)). Tat transactivates the HIV-1 long terminal repeat (LTR) in many human and primate cell types but not in rodent cells. The specific interaction of human Cyclin T1 (hCyclin T1), but not murine Cyclin T1 (mCyclin T1), with HIV-1 Tat protein and TAR RNA to form a multi-component ribonucleoprotein complex is necessary for p-TEFb to mediate a Tat specific and species-restricted activation of HIV-1 transcription (K. Fujinaga et al.). However, hCyclin T1 expression does not induce HIV-1 replication in murine cells (R. Mariani et al., “A block to human immunodeficiency virus type 1 assembly in murine cells,” J. Virol., 74, 3859-3870 (2000)). The Tat-independent activation of HIV-1 provirus transcription has remained unclear in HIV-1-infected rodent cells carrying the receptor genes and hCyclin T1 gene. Host mechanisms regulated or limited by such permissive factors are potential targets for anti-HIV-1 therapy or for construction of a non-human model of primary infection.

HIV-1 infects primate macrophages and activated CD4+ T cells which express major histocompatibility complex (MHC) class II molecules (M. Saifuddin et al., “Cutting Edge: Activation of HIV-1 Transcription by the MHC Class II Transactivator,” J. Immunol., 164, 3941-3945 (2000); M. Saifuddin et al., “Expression of MHC Class II in T cells is associated with increased HIV-1 expression,” Clin. Exp. Immunol., 121, 324-331 (2000)). HIV-1 expression and replication is substantially higher in HLA-DR (human MHC class II) positive T cell lines than in HLA-DR negative ones (M. Saifuddin et al.). A specific transcriptional co-activator, human class II transactivator (hCIITA), regulates MHC class II gene expression. hCIITA binds to the same region as Tat binding site on hCylin T1 and forms the pTEFb complex together with CDK9 for the expression of target genes (S. Kanazawa et al., “Tat competes with CIITA for the binding to p-TEFb and blocks the expression of MHC class II genes in HIV infection,” Immunity, 12, 61-70 (2000)). hCIITA also enhances HIV-1 LTR expression in a variety of human cell types (M. Saifuddin et al.).

It would be desirable to have a suitable in vivo non-primate animal model of HIV-1 infection, since only primates can be used for in vivo studies. This deficiency has hampered the development of immunization and/or therapeutic regimens for AIDS. Thus far, those animal models that have been reported have been unsuitable for studies of HIV-1 infection because they lack the important requirement of supporting HIV-1 replication or virus-induced pathogenesis.

### BRIEF DESCRIPTION OF THE DRAWINGS

FIG. 1 provides a schematic illustration of three transgenes: pUC/CXCR4-CycT1, pUC/CycT1-CCR5, and pUC/CIITA-CD4.

### SUMMARY OF THE INVENTION

The present invention provides rodent animals and cells that produce HIV particles. The rodent animals and cells of the present invention contain and are able to stably express an active portion of one or more of human CD4, a human chemokine co-receptor (such as CXCR4 or CCR5), human cyclin T1, and a human class II transactivator (CIITA), and produce HIV virus particles. The animals and cells of the present invention are able to be infected by and support replication of the HIV virus. Therefore, the present invention provides valuable tools for studying HIV infection. These tools can be used to study methods of treatment applicable to humans and the effectiveness of drug products for preventing and treating HIV disease. Also provided are methods of preparing the transgenic cells and rodent animals of the invention, as well as methods of using them to identify and assay test agents for anti-HIV-1 activity. Also provided are methods and pharmaceutical compositions for treating and preventing HIV infection in a mammal. The rodent animal can be a rat, mouse, or any rodent.

Thus, in a first aspect, the present invention features a rodent cell that replicates the HIV provirus and produces HIV particles. In various embodiments the rodent cell is that of a rat or mouse. The rodent animal cell stably expresses an active portion of one or more of the above proteins. In various embodiments the rodent animal or cell stably expresses a gene or nucleotide sequence coding for one or more of the above proteins, or an active portion of one or more of the above proteins. In preferred embodiments, the rodent cells of the present invention can be infected by the HIV virus, can transcribe and replicate HIV virus, can integrate the HIV provirus into the cell's genome, produce viral proteins, transcribe the HIV provirus, and support virus-induced pathogenesis. In preferred embodiments, the cells produce infectious HIV virus particles.

By the “active portion” of a protein (such as, for example, CD4, a chemokine receptor, cyclin T1, or Class II Transactivator) is meant that portion of the protein without which a cell cannot be recognized by, infected by, and produce HIV virus. In one embodiment, the “active portion” of a protein is preferably the entire native protein. But it can also include any sub-part or variation of the native protein that accomplishes the function of permitting the cell to be recognized and infected by an HIV virus. The active portion of a protein can also be characterized by performing the same function as the native protein and/or having the same activity, with relation to the native protein's role in an HIV virus recognizing and infecting a cell and producing HIV particles. By reference to a protein is meant not only the native, wild type protein, but also any variation of the protein that accomplishes the functions described above. In various embodiments, the protein is a homologue of the native, wild type protein that has at least 60% or 70% or 80% or 90% homology. The active portion of hCyclin T1 and hCIITA include those portions necessary for HIV provirus replication. By a cell or animal “expressing” a protein or peptide is meant that a nucleic acid sequence coding for the protein or peptide is present in the animal or cell and is translated to the protein. Similarly, by “expressing” a gene, nucleotide, or nucleic acid sequence is meant that the sequence is translated into a protein product. By “HIV virus” is meant any human immunodeficiency virus (HIV) that can infect humans. This includes any of HIV-1, HIV-2, HIV-3, or any HIV that can infect humans, and includes primary M-tropic, T-tropic, or dual-tropic viruses. By a cell becoming “infected” by the HIV virus is meant that the virus recognizes receptors on the cell, which direct its tropism to the cell, and viral nucleic acid enters the cell and is incorporated into the genome of the cell. The HIV virus can be primary T-tropic, M-tropic, or dual-tropic viruses, and can be the HIV-1 virus. The “genome” of the cell is the chromosomal DNA of the cell.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (Devereux, J., et al., Nucleic Acids Res. 12(1):387 (1984)) (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Myers and W. Miller (CABIOS, 4:11-17 (1989)) which has been incorporated into the ALIGN program (version 2.0), using a PAM120 weight residue table, a gap length penalty of 12 and a gap penalty of 4.

By “HIV virus particles” is meant the whole, wild type HIV virus or a part of the protein envelope of the HIV virus that can be detected in an assay. In one embodiment, the particle is preferably a whole HIV virus particle capable of infecting and reproducing in a mammalian cell, but can also be a sub-portion of the protein coat of an HIV virus that is not capable of reproducing in a mammalian cell, or even of infecting a living cell. The person of ordinary skill in the art will realize that if a cell produces a portion of the protein envelope of an HIV virus, this can nevertheless be a useful indicia for determining the efficacy of certain anti-HIV drugs on the cell, as the portion can be detected and used as an indicia of infection. By “viable HIV virus particle” is meant an HIV virus particle that can infect a host cell and replicate to produce HIV virus particles, which can in turn infect another host cell, replicate, and produce HIV virus particles.

By “stably expressing” in reference to a protein or a portion thereof (for example a CD4, a chemokine receptor, a cyclin T1, or a Class II transactivator) is meant that the protein or portion of the protein is expressed in at least one progeny cell or animal generation after a nucleotide coding for the protein or portion of the protein is introduced into the parent generation. The parent generation is the first cell or animal that receives the nucleotide. The progeny generation is the result of a cell division and multiplication of the parent cell, or reproduction of the animal to form a progeny generation. Normally the nucleotide coding for the protein or portion of the protein will be introduced into the genome of the parent generation and expressed and passed on to the progeny generation where it is also expressed. The second progeny generation is the result of a cell division or reproduction of the progeny generation. In various embodiments, the nucleotide introduced into the parent generation is expressed in at least one cell or animal of the second progeny generation, or in a majority of cells or animals of the second progeny generation. In other embodiments, the protein will also be stably expressed in the progeny generation, and the second progeny generation. Similarly, by “stably expressing” in reference to a gene, nucleotide, or nucleic acid sequence is meant that the gene, nucleotide, or nucleic acid sequence is introduced into a parent cell and is passed on to at least one progeny cell or animal generation, and that the coding portion of the nucleotide is translated into a protein product. In other embodiments, the gene, nucleotide, or nucleic acid sequence is expressed in a majority of cells or animals of the progeny generation. It can also be stably expressed in at least one cell or animal of the second progeny generation, or a majority of the cells or animals.

In another aspect the present invention provides methods for preparing a rodent animal cell of the present invention. The methods include introducing into a rodent animal cell of the present invention a nucleotide sequence coding for an active portion of one or more of the above proteins. Nucleotides coding for the active portion of one or more of the proteins are incorporated into the genome of the rodent animal cell, and the animal cell stably expresses the active portion of the proteins and can produce HIV virus particles. In preferred embodiments, the animals produce infectious HIV virus particles.

In another aspect, the present invention provides a transgenic rodent animal replicating HIV virus and producing HIV virus particles when infected with HIV. In one embodiment, the animals are capable of replicating HIV virus and producing HIV virus particles when infected with HIV, but are not actively doing so. By “replicating HIV virus” is meant that replicas or copies of the virus are produced, and that the number of viruses is multiplied. The transgenic rodent animal is capable of being infected with the HIV virus and of developing HIV disease, of exhibiting symptoms of HIV infectious disease, of integrating the HIV provirus into the animal's genome, and of supporting virus-induced pathogenesis. The animal can stably express one or more of the above nucleotides coding for one or more of the above proteins or an active portion thereof. Thus, the transgenic animal expresses at least one human protein or part thereof. In preferred embodiments, the rodent animals can transcribe and replicate HIV virus, produce viral proteins, and transcribe the HIV provirus. By “developing HIV disease” is meant that the organism shows clinical signs of HIV disease, as recognized by those of ordinary skill in the art. The clinical signs include any of the following signs in the presence of, and because of, the HIV virus: loss of weight, pyrexia, diarrhea, systemic lymph node swelling, pancytopenia, anemia, opportunistic infectious diseases (such as pneumocystis carinii pneumania, cryptosporidiosis toxoplasmosis, isosporiasis, strongyloidiasis, candidiasis, cryptococcosis, histoplasmosis, mycobacterium avium or kansasii infections, cytomegalovirus infection, herpes simplex infection, progressive multifocal leukoencephalopathy due to papovavirus infection of central nervous system), Kaposi's sarcoma, non-Hodgkin lymphom, encepholopathy, demetia, hyperplasia, splenomegaly, lymphadenopathy, pulmonary lymphoid infiltrates, and growth retardation.

In other embodiments, other clinical signs may also indicate that an organism has developed HIV disease. The signs described above are also the signs of virus-induced pathogenesis.

In another aspect, the present invention provides methods for preparing a transgenic rodent animal of the present invention. The methods include introducing into an embryonic cell of a rodent animal one or more of the above nucleotides and developing the embryonic cell to obtain a transgenic rodent animal of the present invention. The nucleotides can be introduced into the embryonic cell on one or more plasmids, such as pUC18 and pGEM-T Easy, or by methods known to those of ordinary skill in the art. In preferred embodiments, the animals of the present invention can perform one or more of the following: be infected by the HIV virus, transcribe the HIV provirus, replicate the HIV virus, and transcribe and translate HIV viral proteins. By an “embryonic cell” is meant a cell that is a precursor cell to other types of cells.

In another aspect the present invention provides methods for assaying for anti-HIV activity of a test agent. The methods include contacting a transgenic rodent animal cell of the present invention with a test agent. The cell can be infected with the HIV virus and the level of HIV RNA, circulating virus particles, CD4+ T-lymphocytes, viral proteins, and antibodies against viral proteins present in the cell monitored to determine the anti-HIV activity of the test agent. In another aspect, the test agent can be administered to a transgenic rodent animal of the present invention, which has been previously infected with the HIV virus, is producing HIV virus, and is exhibiting symptoms of HIV infectious disease. The level in the blood of the animal one or more indices can be monitored.

By “anti-HIV activity” of a test agent is meant a reduction in the level or presence of one or more indicia of an HIV infection. The indicia may be a reduction in the level of any of the following in a cell or organism: the production of HIV particles, HIV RNA in the cell or animal, circulating virus particles, CD4+ T-lymphocytes, viral proteins, and antibodies against viral proteins, all relative to the level of the indicia in the absence of the agent. By “monitoring the level of” is meant obtaining at least one data point indicating the level of the relevant indicator. The monitoring will frequently be to determine whether or not the level increases, but the monitoring may also include gathering data points indicating a decrease in the level of the indicator. An “indicator” can be, but is not limited to, HIV RNA, circulating virus particles, CD4+ T lymphocytes, viral proteins, and antibodies against viral proteins. A change in these indicators can be an indication of HIV infection in a mammalian cell.

In another aspect, the present invention provides methods for identifying an agent having anti-HIV activity. The methods include contacting a test agent with a transgenic animal or cell of the present invention that is infected with the HIV virus and is producing HIV virus particles, and monitoring the level of one or more of the above indicia in the animal or cell. Thus, an agent having anti-HIV activity can be identified. In another aspect the methods can include monitoring the level of one or more of the above indicia, and infecting the animal cell with HIV after contacting the animal or cell with the test agent. In preferred embodiments the contacting can be including the test agent in the growth media of the cell.

In another aspect the present invention provides methods for treating symptoms of HIV infection in a mammal. The methods include administering a test agent to a transgenic rodent animal of the present invention that is producing HIV virus and is exhibiting symptoms of HIV infectious disease, monitoring one or more symptoms associated with HIV infection in the animal, and identifying an agent capable of alleviating one or more symptoms of HIV infection as an agent having an anti-HIV activity. An affected mammal can then be treated with the agent identified. The compound can be administered by any means including, but not limited to, intraperitoneally, intravenously, and orally. By “symptoms of HIV disease” in an animal is meant the level in the blood of the animal of any of one or more of the above indicia of an HIV infection or clinical signs of developing HIV disease described above. In another aspect the present invention provides methods for treating and preventing symptoms of HIV infection in a mammal using the above methods.

In another aspect the present invention provides agents identified by the methods of the present invention. Also provided are pharmaceutical compositions for preventing or treating HIV-1 infectious disease, or compositions having anti-HIV activity that contain one or more agents identified by the methods of the present invention. The present invention also provides uses of the cells and animals of the present invention, which include uses of identifying agents having anti-HIV activity.

The summary of the invention described above is not limiting and other features and advantages of the invention will be apparent from the following detailed description of the preferred embodiments, as well as from the claims.

### DETAILED DESCRIPTION OF THE INVENTION

The exacting requirements for HIV entry into a cell strongly influence which cells the HIV-1 virus is directed toward, and present a major barrier to HIV infection in non-humans (W. A. Paxton et al., “Relative resistance to HIV-1 infection of CD4 lymphocytes from persons who remain uninfected despite multiple high-risk sexual exposures,” Nat. Med. 2, 412-417 (1996); H. Choe et al., “The beta-chemokine receptors CCR3 and CCR5 facilitate infection by primary HIV-1 isolates,” Cell 85, 1135-1148 (1996); Y. Feng et al., “HIV entry cofactor: Functions1 cDNA cloning of a seven-transmembrane, G protein-coupled receptor,” Science 272, 872-877 (1996)). Although human CD4 is a marker for helper T cells and a principal receptor for HIV entry to cells, chemokine receptors are also required as co-receptors (A. G. Dalgeish et al., “The CD4 (T4) antigen is an essential component of the receptor for the AIDS retrovirus,” Nature, 312, 763-767 (1984)). CCR5 is the principal co-receptor for primary macrophage (M-tropic) HIV-1 strains (H. Choe et al.), while CXCR4 supports infection of T-tropic HIV strains (A. G. Dalgeish et al.). These receptors are described in detail in “International Union of Pharmacology: XXII. Nomenclature for Chemokine Receptors,” Murphy et al., Pharmacological Reviews, Vo. 52, No. 1 (2000), which is hereby incorporated by reference in its entirety. We have found that these molecules, which are present on the cell surface, determine not only cell tropism but also species tropism of HIV-1 infection. Structural differences of CD4, CXCR4 and CCR5 between human and other mammals, including rodents, are major factors for species-specific infection of HIV-1. Co-expression of hCD4 and hCXCR4 or hCCR5 in rat W31 cells allows for HIV-1 entry and integration of the complete provirus into the host genome, but does not support the transcription of HIV-1 provirus and viral protein production in rat cells. We have discovered that in order for a rodent cell or animal to support HIV replication, produce HIV-1 particles, and provide a most useful model of virus-induced pathogenesis, it is necessary to express not only hCD4 and a human chemokine receptor, but also a human cyclin T1 and a human class II transactivator (hCIITA). The person of ordinary skill in the art will realize that expression of the entire nucleic acid coding sequence or protein may not be necessary for the production of viral particles, but that expression of an active portion is sufficient.

Tat, a regulatory protein encoded by HIV-1, is a potent activator of the viral LTR promoter and is required for virus replication (K. A. Jones et al., “Taking a new TAK on tat transactivation,” Genes Dev. 11, 2593-2599 (1997); B. R. Cullen, “HIV-1 auxiliary proteins: Making connections in a dying cell,” Cell 93, 685-692 (1998)). In the absence of Tat, the HIV-1 LTR generates short or non-processive transcripts (K. A. Jones et al.). Transcription of HIV-1 stimulated by Tat requires specific cellular co-factor(s) present in many human and primate cells but not in rodent cells (K. A. Jones et al., “Control of RNA initiation and elongation at the HIV-1 promoter,” Annu. Rev. Biochem. 63, 717-743 (1994)). The presence of Tat results in a large increase in expression of the entire HIV-1 genome (K. A. Jones et al.). Tat-dependent transcription requires p-TEFb. The p-TEFb complex, composed of CDK9 and Cyclin T1, is a general transcription elongation factor that then specifically binds to the activation domain of Tat, and phosphorylates the carboxyl-terminal domain of RNA polymerase II (P. D. Bieniasz et al., “Recruitment of a protein complex containing Tat and cyclin T1 to TAR governs the species specificity of HIV-1 Tat,” EMBO J. 17, 7056-7065 (1998); N. Marshall et al., “Control of RNA polymerase II elongation potential by a novel carboxyl-terminal domain kinase,” J. Biol. Chem. 271, 27176-27183 (1996)). Cyclin T1 interacts with Tat and assists in recruiting the p-TEFb complex to the HIV-1 promoter (P. Wei et al.; M. E. Gaber et al., “The interaction between HIV-1 Tat and human cyclin T1 requires zinc and a critical cysteines residue that is not conserved in the murine CycT1 protein,” Genes Dev. 12, 3512-3527 (1998)). Human Cyclin T1 (hCyclin T1) but not murine Cyclin T1 (mCyclin T1) enhances Tat-mediated transactivation of the HIV-1 LTR, since mCyclin T1 has a tyrosine in place of a cysteine at position 261 and is unable to support Tat transactivation (P. D. Bieniasz et al.). hCyclin T1 expression does not support virus replication in rat and mouse cells carrying a full HIV-1 provirus genome, (R. Mariani et al.) although some structural or non-processing transcripts are induced. Thus, HIV-1 replication in rodent cells requires other species specific factor(s). By fusion with human peripheral blood mononuclear cells (hPBMC) but not rat cells (rPBMC), the hCyclin T1-transfected hCD4-hCXCR4_{SF33}, hCD4-hCCR5_{JRFL} or hCD4-hCCR5_{JRCSF} cells produced p24, an event was not detected in culture prior to the fusion.

The HIV-1 virion acquires host cell proteins, including MHC class I and II antigens, as ligands to increase infectivity of HIV-1 by co-expression together with viral envelope protein on the surface of the virion when they bud from the host cell membrane (M. J. Tremblay et al., “The acquisition of host-encoded proteins by nascent HIV-1.” Immunol. Today 19, 346-351 (1998); A. S. Fauci, “Host factors and the pathogenesis of HIV-induced disease,” Nature 384, 529-534 (1996)). HIV-1 infects both macrophages and activated CD4 cells, and both cell types express MHC class II antigens (M. Saifuddin et al. J. Immunol.; M. Saifuddin et al., Clin. Exp. Immunol.). Immunization with common recall MHC antigens results in a transient increment in plasma viremia in HIV-infected individuals (S. K. Stanley et al., “Effect of immunization with a common recall antigen on viral expression in patients infected with human immunodeficiency virus type 1,” N. Engl. J. Med. 334, 1222-1230 (1996); M. A. Ostrowski et al., “Increased in vitro tetanus-induced production of HIV type 1 following in vivo immunization of HIV type 1-infected individuals with tetanus toxoid,” AIDS Res. Hum. Retroviruses 13, 473-480 (1997); S. I. Staprans et al., “Activation of virus replication after vaccination of HIV-1 infected individuals,” J. Exp. Med. 182, 1727-1737 (1995); H. Moriuchi et al., “Induction of HIV-1 replication by allogenic stimulation,” J. Immunol. 162, 7543-7548 (1995)). MHC class II gene expression is regulated by CIITA. In addition, CIITA has an acidic N-terminal activation domain and a C-terminal domain important for protein-protein interactions (H. Zhou et al., “Human MHC class II gene transcription directed by the carboxyl terminus of CIITA, one of the defective gene in type II MHC combined immune deficiency,” Immunity 2, 545-553 (1995); K. C. Chin et al., “Importance of acidic, proline/serine/threonine-rich, and GTP-binding regions in the major histocompatibility complex class II transactivator: generation of transdominant-negative mutants,” Proc. Natl. Acad. Sci. USA 94, 2501-2506 (1997)). Stable expression of hCIITA increased the provirus expression and LTR promoter activity in 293 and HeLa-T4 cells (M. Saifuddin et al., J. Immunol.). hCIITA binds to the same region of Tat binding site on the hCyclin T1, and Tat competes for the binding of hCIITA to the hCyclin T1 N-terminal sequences and blocks the expression of MHC class II genes (S. Kanazawa et al.), suggesting that hCIITA recruits p-TEFb by binding hCyclin T1 and promotes HIV-1 expression in Tat independent activation. hCIITA which has about 70% homology to murine CIITA may also act a potential species-specific co-factor for HIV-1 replication. Indeed, double transfection of hCIITA and hCyclin T1 induced the replication of infectious HIV-1 in W31 cells carrying the HIV-1 provirus.

The inventors have discovered that hCIITA co-functions with hCyclin T1 to overcome the suppression of HIV-1 provirus replication in rat cells in a post entry and Tat-independent manner. Thus, co-expression of hCD4, human chemokine receptors, hCyclin T1 and hCIITA in rats and other mammals breaks through the species barrier of HIV-1 infection and replication in vivo, and allows the creation of an animal model of HIV-1 infection, using transgenic techniques. The proteins CD4, CCR5, CXCR4, human cyclin T1, and human Class II transactivator are well known in the art and literature is available regarding their characterization. For example, CCR5 was characterized by C. J. Raport et al., J. Biol Chem (1996) 271:29, pp. 17161-66 and other sources, and genotyping assays are available that disclose wild type and mutated probes, e.g., J. W. Romano et al., Clin Diagn Lab Immunol. (1999) 6:6 pp. 959-65; antibodies have been raised to CD4 (Sekigawa et al., Clin Immunol Immunopathol (1991) 58:1 pp. 145-53; CXCR4 has been characterized by many investigators, e.g., Hesselgesser et al., J Immunol 1998 160:2 pp. 877-83, and probes for CXCR4 are available in the literature, e.g., Zou et al., Nature, Vol. 393, pp. 595-99 (1998); Cyclin T1 is described, for example, in A. DeLuca et al., J Histochem Cytochem (2001) 49:6 pp. 685-92; and Class II transactivator has been described, for example, by C. H. Chang et al., J Exp Med (1995) 181:2 pp. 765-7 and probes are available, e.g., J. F. Piskurich et al., Nature Immunology, Vol. 1, No. 6, pp. 526-32 (2000).

The person of ordinary skill in the art will appreciate that the present invention provides valuable tools for studying HIV infection, for identifying agents that have anti-HIV activity, and for identifying new methods of treating HIV. The rodent cells of the present invention are useful for determining the anti-HIV activity of test agents. Test agents can be identified that may have anti-HIV activity, and these agents administered to a cell of the invention and the cell's reaction to such exposure determined. Test agents will be identified that prevent or impede the ability of HIV virus to infect the cell. Agents will also be identified that prevent a cell that has already been infected by the HIV virus from being destroyed by the virus.

Similarly, the transgenic rodent animals of the invention are also useful for studying HIV infection at many levels. Test agents that may have the ability to prevent a cell from being infected by the HIV virus. Agents will be identified that prevent the animals from becoming infected by the HIV virus. Also, agents will be identified that ameliorate symptoms of HIV infection in an animal that has been previously infected by the HIV virus, thus enabling the animal to survive, display normal behavior, and live out a normal life cycle even though it is infected with the HIV virus. Any of the above-identified agents can be included in pharmaceutical compositions for treating HIV infection.

### EXAMPLE 1

### Expression Constructs for hCD4, hCXCR4, hCCR5h, hCyclin T1 and hCIITA

An hCD4 expression construct provided by H. Karasuyama (Tokyo Metropolitan Institute of Medical Science, Tokyo, Japan) (Y. Yamamura et al., “Infection of human CD4+ rabbit cells with HIV-1 possibility of the rabbit as a model for HIV-1 infection,” Int. Immunol. 3, 1183-1187 (1991) was used. Of course the person of ordinary skill will realize that many other expression systems are available for expressing CD4, e.g., M. G. Harris et al. (1994), J Mol Biol 241, 136-142 (Repository Reference: ARP281.1-2); B. Chackerian et al. (1995), Virology 213: 386-394 (Repository Reference: ARP287), or from The NIH AIDS Research and Reference Reagent Program (Repository Reference: ARP242).

cDNAs of hCXCR4, hCCR5 hCyclin T1 and hCIITA were cloned by reverse transcriptase PCR (RT-PCR). Total RNA was isolated from hPBMC, using the ISOGEN® kit (Nippon Gene Co., Toyama, Japan) and applied to the RT reaction with oligo-(dT) primer using a SUPERSCRIPT RT® (GIBCO-BRL, Rockville, Md.). The cDNA products were amplified with gene specific primers designed according to each DNA sequence of hCCR5, hCXCR4, hCyclin T1 and hCIITA (Table 1), and each amplified cDNA was subcloned into pUC18 or pGEM-T EASY® (Promega, Madison, Wis.), using appropriate restriction enzymes or TA cloning. Each cloned cDNA was confirmed by DNA sequencing to be the correct DNA sequence of each gene. After digestion with appropriate restriction enzymes, each cDNA was ligated into the multicloning site of expression vectors (Table 1).

T-vectors are linear-blunt-ended plasmids with dT's added on by Taq polymerase. Many protocols are available for making T vectors. The following is one example: Digest pBSKS (bluescript) with Eco R5; precipitate with 3M NaOAc and 2.5 volumes EtOH, −20° C. 1 hr, centrifuge 20-30 minutes; wash with 70% EtOH, dry. Bring up in 10 ul of 0.1×T.E. Add only dTTP to final concentration of 2 mM. A recipe is as follows: 10 μl Eco R5 digested plasmid; 5 ul 100 mM dTTP, 2 mM; 5 μl 10×Taq buffer, 1×; 4 μl 25 mM MgCl₂, 2 mM; 1 μl Taq polymerase; 25 μl dH₂O; Final volume is 50 μl, Add a drop of oil to top of reaction mix. Use a PCR machine for reaction, 72° C. 3 hours; clean up reaction—either phenol, phenol/chloroform, chloroform, or ethanol.

Many TA cloning techniques are known in the art (e.g., Clark, J. M. (1988) Nuc. Acids Res. 16:9677-9686. Gahm, S. J. et al. (1991) Proc. Natl. Acad. Sci.88:10267-10271. Mead, D. et al. (1991) Bio/Technology9:657-663. The following protocol is only one example: Initial mixture 5 μl dd H₂O, 1 μl PCR product, 1 μl ligation buffer, 2 μl TA vector (add second to last), 1 μl ligase (add last); Incubate overnight at 15° C., Electroporation: Chill cuvettes on ice before starting; Set out electrocompetent JS5 cells to thaw on ice. Dry one 100 mg/ml AMP plate for each sample, add 40 μl of xgal and 40 μl of IPTG to each plate, Add 500 μl of SOC or SOB media to each 5 ml snap cap tube; Heat ligations at 65° C. for 5-7 minutes, set at room temperature; Dilute ligations 1:25 in water (this dilution is optional, 1 μl of ligation has been used effectively); Add 30 μl of competent cells to each chilled cuvette. Add 1 μl of ligation into chilled cuvettes. Tap several times to mix. Turn electroporator on. Put the cuvette in the electroporator. Immediately, remove the sample from the cuvette and put into the tube with the SOC or SOB. Flick the tube to resuspend. Incubate in the shaker at 37° C. for 30 minutes to 1 hour. Repeat above for each ligation. After the incubation, plate 100 μl on one plate containing the xgal and IPTG. Invert and incubate overnight at 37 ° C. Check for positives by PCR a. with internal PCR primers b. with forward and reverse primer. Pick white colonies into PCR mix, then streak on 2×YT & Amp to create stock. Incubate plate at 37° C. overnight.

Procedures for performing RT-PCR are well known in the art and are publicly available. For example, the following protocol may be used, which performs cDNA synthesis on DYNABEADS® oligo(dT)₂₅ (Dynal Biotech, Oslo, Norway) magnetic beads. Dissolve RNA (30 μg) in 10 μl H₂O, add 20 μl TE/1M KCl; a) Place 100 μl DYNABEADS® oligo(dT)₂₅ (5 mg/ml) in a 0.5 ml tube. b) Bind beads. c) Remove liquid. d) Add 100 μl TE/1M KCl. e) Wash. f) Bind beads. g) Remove liquid. Add RNA to beads. Heat to 70° C. for 2 min and cool slowly to RT for 10 min. Bind beads. Remove liquid. Resuspend beads in 2.5 μl Buffer A (200 mM Tris-HCl, pH 8.3,1.0 M KCl), 2.5 μl Buffer B (30 mM MgCl₂. and 15 mM MnSO₄), 20.0 μl dNTPs (2.5 mM each), 1.0 μl 32P-dCTP (5 uCi), 1.0 μl RNasin-Pharmacia, 2.0 μl SuperScript II RT (200 U/μl) (Gibco BRL #18064-014), 5.0 μl Retrotherm RT (1 U/μl) (Epicentre Technologies #R19250), 16.0 μl H₂O. Remove 1 μl of reaction. This represents total ³²P counts for use in calculating the amount of cDNA synthesized. Heat at 40° C. for 30 min. Heat at 70° C. for 1 hr. Bind beads and remove all liquid. Wash beads with 100 μl TE, bind beads, remove liquid. Resuspend beads in 100 μl TE. Count 1 μl of beads to calculate the amount of cDNA synthesized. Use 1 μl of beads per PCR. For additional information, the following references can be consulted. Raineri, I., Moroni, C. and Senn, H. P. (1991). Improved efficiency for single-sided PCR by creating a reusable pool of first-strand cDNA coupled to a solid phase. Nucleic Acids Res. 19: 4010-20; Rodriguez, I. R., Mazuruk, K.,Schoen, T. J. and Chader, J. G. (1994). Structural analysis of the human hydroxyindole-O-methyltransferase gene: presence of two distinct promoters. J. Biol. Chem. 269, 31969-31977; Rodriguez, I. R. and Chader, G. J., A novel method for the isolation of tissue-specific genes, Nucleic Acids Res., 20 (1992) 3528; Schoen, T. J., Mazuruk, K., Chader G. J., and Rodriguez, I. R. Isolation of candidate genes for macular degeneration using an improved solid-phase subtractive technique. Biochem. Biophys. Res. Commun., 213 (1995) 181-8; “Detection and quantitative determination of RNA and DNA WO90/06042; “In vitro mutagenesis,” WO90/06043, “Process for producing cDNA,” U.S. Pat. No. 5,759,820, “Nucleic acid probes/Oligonucleotide-linked magnetic particles and uses thereof,” U.S. Pat. No. 5,512,439, “Separator device for magnetic particles,” WO90/14891; “Cloning method and kit,” U.S. Pat. No. 5,525,493, “Method of separating haemopoictic progenitor cells,” WO91/09938; “Antigen/anti-antigen cleavage,” U.S. Pat. No. 5,429,927, “Isolation of nucleic acid,” WO96/18731; “Method for activating a reversibly inactivated immobilized enzyme by release from an immobilising moiety and its use in nucleic acid amplific,” WO96/35779; “The use of modular oligonucleotides as probes or primers in nucleic acid based assay,” WO98/13522; “Biomolecules comprising an elastomeric peptide,” WO99/11661; “Method of isolation primer extension products with modular oligonucleotides,” WO00/15842; “Nucleic Acid Probes,” U.S. Pat. No. 5,512,439; “Process for producing cDNA,” U.S. Pat. No. 5,759,820; “Detection and Quantitative Determination of RNA and DNA,” EP 444120; and “In vitro Mutagenesis,” EP 448609.

TABLE 1
Gene Specific Primers for RT-PCR Cloning and
Expression Vectors Used for Transfection

| | Expres- | |
|---|---|---|
| | sion | |
| Gene | Vector | Sequences (5′ 3′) |
| hCCR5 | pcDNA3.1/ | (+)^{b} CTGAGACATCCGTTCCCCTACAAG |
| | Zeo(−) | AAACTC |
| | | (+) GATACCTCCCTCCTTCCCATCCTTAC |
| | | GAA |
| hCXCR4 | pcDNA3.1/ | (+) AGTGCTGCAGTAGCCACCGCATCTG |
| | Zeo(−) | (−) TAGATCTGTGTTAGCTGGAGTGAGG |
| | | GCTTG |
| hCyclin T1 | pEFb6.0/ | (+) GTCTGATGAGGATCCATGGAGGGAG |
| | pEYFP-cl | AGAGGAAGAACAA |
| | | (−) GTCTAGTAGTCTAGATTACTTAGGA |
| | | AGGGGTGGAAGT |
| hCIITA | pEGFP-cl | (+) CTGCCTGGCTGGGATTCCTACACAA |
| | | TGCGT |
| | | (−) ATGCCTGTCCAGAGCACAGCTGGGA |
| | | TCATC |

^{a}GenBank accession number: hCCR5, U54994; hCXCR4, Y14739; hCyclin T1, AF048730; hCIITA, NM000246.
^{b}(+) means sense primer and (−) means antisense primer

### EXAMPLE 2

### Transfection

The resultant expression constructs were transfected into a rat fibroblast cell line, W31 (provided by N. Sato, Sapporo Medical University, Sapporo, Japan), using the SuperFect™ Transfection Reagent (Qiagen, Valencia, Calif.) following the manufacturer's instructions. W31 are available from the Division of Cancer Pathobiology, Institute for Genetic Medicine, Hokkaido University, Japan. But the person of ordinary skill will realize that any of the rat or mouse fibroblast cell lines will be useful, such as Rat2, NRK-49F, Rat1-R12, 3T3, A9, and NCTC clone 2472, all of which are available through various vendors including the ATCC. SuperFect™ consists of activated-dendrimer molecules with a defined spherical architecture (M. X. Tang et al., (1996) In vitro gene delivery by degraded polyamidoamine dendrimers, Bioconjugate Chem., 7, 703). The transfectants were selected and maintained in RPMI-1640 medium supplemented with 10% fetal calf serum (FCS), 400 μg/ml G418 (GIBCO-BRL), 40 μg/ml Zeocin™ (phleomycin D1, Invitrogen, Carlsbad, Cali.) and/or 5 μg/ml Blasticidin (Merck Index, 12, 1350; Kimura, M., et al. (1994), Calbiochem-Novabiochem Co., San Diego, Calif.). Stable transfectants with high levels of transgene expression were cloned by a standard limiting dilution method and the expression levels of each transgene were confirmed by the flow cyometry, as described below.

### EXAMPLE 3

### Flow Cytometry

Mouse anti-CCR5 (clones of 2D7/CCR5, PharMingen, San Diego, Calif.) or anti-hCXCR4 (12G5, PharMingen) monoclonal antibodies were used as first antibodies and a fluorescein isothiocyanate (FITC)-conjugated rabbit anti-mouse IgG antibody as a second antibody. Anti-CCR5 was purified from tissue culture supernatant by Protein G affinity chromatography and conjugated with FITC under optimum conditions. The anti-hCCR5 antibodies were mouse IgG₂ₐ, κ, FITC-conjugated antibody. (Wu, L., “CCR5 levels and expression pattern correlate with infectivity by macrophage-tropic HIV-1 in vitro” J. Exp. Med. 185:1681 (1997)). Phycoerythrin (PE)-conjugated anti-hCD4 monoclonal antibody (DAKO, Glostrup, Denmark) was used for direct staining of hCD4. The cells were analyzed using FACScan (BD Bioscience, San Jose, Calif.). Normal mouse IgG as a first antibody or PE-labeled mouse IgG as a direct staining served as negative controls.

### EXAMPLE 4

### HIV Infection

Primary tropic (SF33) and M-tropic (JRFL and JRCSF, provided by Y. Koyanagi, Tohoku University, Sendai, Japan) HIV-1 isolates were used. The person of ordinary skill in the art will realize that other isolates can be used as well, and these are presented as examples. After treatment with 40 μg/ml of DEAE dextran, transfectants were washed with phosphate buffered saline (PBS) and incubated with each HIV-1 isolate (about 100 TCID₅₀/ml) for 2 h at 37° C. The transfectants were then washed 3 times at each 24 h interval by treatment with a trypsin-EDTA solution, the HIV-1 infected cells were cultured and harvested several times, at intervals to determine the DNA or mRNA of HIV-1 provirus, using a PCR amplified kit for HIV-1 (ABBOTT, Wiesbaden-Delkenheim, Germany). To investigate the production of infectious HIV-1 virus in the HIV-1 infected W31 cells, supernatant from the hCD4-hCXCR4_{SF33} cells at 2 days after the double transfection of hCIITA and hCyclin T1 was harvested and added to the Phytohemagglutinin-activated hPBMC culture. (J. Frenster, “Phytohemagglutinin-Activated Autochthonous Lymphocytes for Systemic Immunotherapy of Human Neoplasms” Annals of the New York Academy of Science, Vol.277: pp.45-51(1976); K. Ozato et al, “Pretreatment of murine thymocytes by PHA inhibits the binding of 3H-concanavalin-A” J. Immunol. 115: 339-344 (1975); M. J. Bevan, “Cytotoxic effects of antigen- and mitogen-induced T-cells on various targets,” J. Immunol. 114: 559-565 (1975)). After an overnight incubation, the hPBMC was washed with phosphate buffered saline (PBS) and cultured with 5% of human interleukin-2 (Hemagen Diagnostic Inc., Columbia, Md.), and p24 in the culture medium was measured at every 4 days.

### EXAMPLE 5

### PCR and RT-PCR

To detect the provirus genome, genomic DNAs from HIV-1-infected transfectants after the infection were amplified by PCR, using HIV-1 specific serial primer pairs for LTR (SK29/SK30) (M. C. Psallidopoulos et al., “Integrated proviral human immunodeficiency virus type 1 is present in CD4+ peripheral blood lymphocytes in healthy seropositive individuals,” J. Virol. 63, 4626-4631 (1989), gag (LAV1/LAV2, Genset, Paris, France) pol (HIV160/HIV161) V. Courgnaud et al., “Frequent and early utero HIV-1 infection,” AIDS Res. Hum. Retroviruses 7, 337-341 (1991), vif((HIV156/HIV157) D. Y. Kwoh et al., “Transcription-based amplification system and detection of amplified human immunodeficiency virus type 1 with a bead-based sandwich hybridization format,” Proc. Natl. Acad. Sci. USA 86, 1173-1177 (1989)), tat (HIV146/HIV148, Genset), env (HIV104/HIV105 (J. Jason et al., “Prevalence of human immunodeficiency virus type 1 DNA in hemophilic men and their sex partners,” Hemophilia-AIDS Collaborative Study Group, J. Infect. Dis. 160, 789-794 (1989)), SK68/SK69 (F. A. Plummer et al. “Detection of human immunodeficiency virus type 1 (HIV-1) in genital ulcer exudate of HIV-1 infected men by culture and gene amplification,” J. Infect. Dis. 161, 810-811 (1990)), env/tat/rev (HIV179/HIV180) (H. Steuler et al., “Distinct populations of human immunodeficiency virus type 1 in blood and cerebrospinal fluid,” AIDS Res. Hum. Retroviruses 8, 53-59 (1992)), and nef(HIV104/HIV141) (G. J. Murakawa et al., “Direct detection of HIV-1 RNA from AIDS and ARC patient samples,” DNA 7, 287-295 (1988)), according to the conditions described for each reference of primers. RT-PCR led to detection of mRNA expression of the provirus. The DNase treated total RNAs from the infected cells were transcribed by a M-MLV reverse transcriptase (RT) (GIBCO-BRL) using a random primer set (Takara, Kyoto, Japan) and amplified by PCR, using the above primer pairs.

### EXAMPLE 6

### Southern Blot

Genomic DNAs from each infected cells were digested with Sma I and Stu I and separated on a 0.8% agarose gel by electrophoresis. After transfer to a nylon membrane hybridization with ³²P-radioactivity labeled HIV-1 DNA probe was performed. The PCR product from env/tat region (677 bp) was used as a probe. Procedures from hybridization to autoradiography were performed according standard methods.

### EXAMPLE 7

### Detection of p24

p24 in the culture medium or cell lysates was determined by an enzyme-linked immunosobent assay. For preparing cell lysates, cells were lysed by a virus disruption buffer (0.5M Tris-HCl (pH 7.8), 0.15 mg/ml Dithiothriotol and 0.1% Triton-X100) and centrifuged at 15,000 rpm for 15 min. The collected supernatant was served as the cell lysates. Many p24 ELISA assays are known in the art, e.g., U. Wienhues, et al., “Boehringer Mannheim modular test concepts in HIV and hepatitis immunoassays,” Clin Biochem (1993) August; 26 (4): 295-99; S. Laal et al., “A rapid, automated microtiter assay for measuring neutralization of HIV-1,” AIDS Res. Hum Retroviruses (1993) August; 9(8): 781-5; J. Schupbach et al., “Antiretroviral treatment monitoring with an improved HIV-1 p24 antigen test: an inexpensive alternative to tests for viral RNA,” J. Med. Virol. (2001), October 65(2): 225-32; S. Chandwani, “Early diagnosis of human immunodeficiency virus type 1-infected infants by plasma p24 antigen assay after imune complex dissociation,” Pediatr Infect Dis J. (1993) January; 12(1):96-7; E. B. Walter, “Enhanced p24 antigen detection in sera from human immunodeficiency virus-infected children,” Pediatr Infect Dis J. (1993) January; 12(1):94-6.

### EXAMPLE 8

### Cell Fusion

The HIV-1-infected cells with hCyclin T1 transfection were gently mixed with phytohemagglitinin (PHA, 3 μg/ml)-activated hPBMC or rPBMC (10 (M. Saifuddin et al.) cells of each), and centrifuged at 1500 rpm for 5 min. After completely removing the supernatant, 0.5 ml of pre-warmed polyethylene glycol 1500 was added drop by drop to the cell mixture, then left for 90 sec. The reaction was stopped by gradually adding of pre-warmed RPMI medium. After centrifugation at 1500 rpm for 5 min., the fused cells were gently re-suspended in RPMI-1640 medium supplemented with 10% Fetal Calf Serum and plated out on a petri-dish.

### EXAMPLE 9

### Preparation of a Transgenic Animal

Construction of the Transgenes

To produce a transgenic rat carrying four HIV-1 infection related human genes, three transgene constructs were prepared. The pX cDNA insert was removed from the pH2/tax.rex expression construct (Yamada S, Ikeda H, Yamazaki H, Shikishima H, Kikuchi K, Wakisaka A, Kasai N, Shimotohno K, Yoshiki T (1995) “Cytokine-producing mammary carcinomas in transgenic rats carrying the pX gene of human T-lymphotropic virus type I,” Cancer Res 55: 2524-2527), contained mouse H-2K^{d} promoter lesion, HTLV-I pX cDNA and SV40 polyA signal, with EcoR1 digestion. Cloned full length hCXCR4, hCCR5, hCIITA or hCyclin T1 cDNAs were ligated into the site. After digestion with appropriate restriction enzymes (Kpn I and Sal I), each expression cassette, including the H-2K^{d} promoter, hCXCR4, hCCR5, hCIITA or hCyclin T1 cDNAs, and SV40 polyA signal sequence, was used for construction of the three transgenes described below.

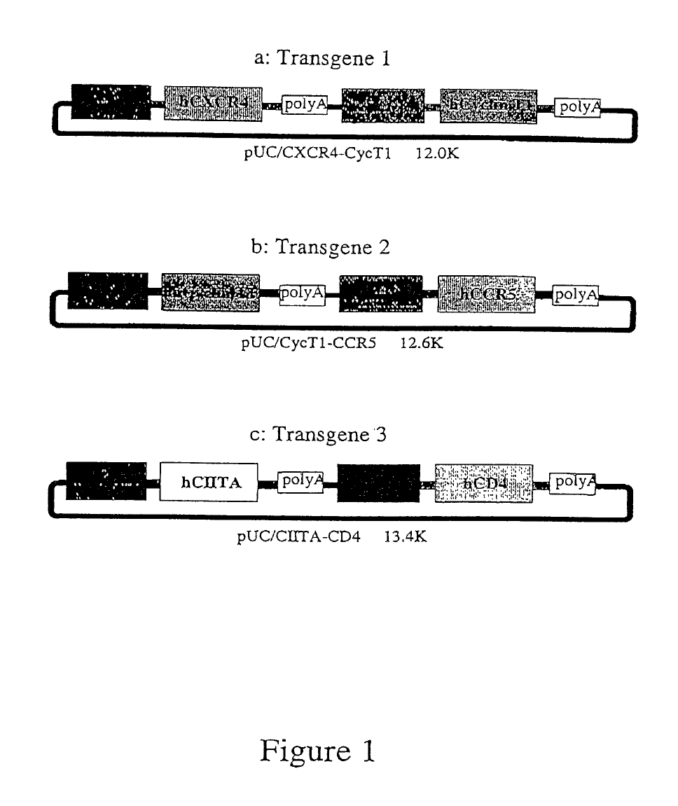

Transgene 1: First, pUC119 with a hCyclin T1 expression unit (pUC/CycT1) was produced by insertion of the expression cassette of hCyclin T1 between the Bam HI and Sal I sites on the multicloning site of the pUC119 vector. The hCXCR4 expression cassette was then ligated into the Kpn I site of pUC/CycT1. (FIG. 1a)

Transgene 2: For insertion of the hCCR5 expression cassette into pUC/CycT1, a multicloning sequence was inserted between the Sal I and Hind III sites behind the hCyclin T1 expression unit of pUC/CycT1. The hCCR5 expression cassette was then inserted between the Kpn I and Sal I sites on the multicloning site. (FIG. 1b)

Transgene 3: The human CD4 promoter (Hanna Z, Simard C, Laperriere A, Jolicoeur P (1994) “Specific expression of the human CD4 gene in mature CD4+ CD8− and immature CD4+ CD8+ T cells and in macrophages of transgenic mice,” Mol Cell Biol 14, 1084-1094; GenBank accession number is S68043) for hCD4 cDNA expression was cloned from genomic DNA of human peripheral blood mononuclear cells using the PCR cloning method.

After confirming the DNA sequence of the clone, the mouse H-2K^{d} promoter in pH2/tax.rex was replaced by the cloned CD4 promoter. The pX cDNA insert was then replaced by a cloned full-length hCD4 cDNA. The hCD4 expression cassette with the human CD4 promoter was obtained by Kpn I and Sal I digestion. The hCIITA expression cassette and hCD4 expression cassette were ligated together between Eco RI and Sal I sites on the multicloning site of pUC119. (FIG. 1c)

Microinjection

After linearization of each transgene, mixtures of Transgene 1 (pUC/CXCR4-CycT1) and 3 (pUC/CIITA-CD4) for T-tropic HIV-1 infection or of Transgene 2 (pUC/CycT1-CCR5) and 3 (pUC/CIITA-CD4) for M-tropic HIV-1 infection are microinjected into fertilized ova of female rats according to methods known in the art (e.g., Yamada et al.; Yamazaki H, Ikeda H, Ishizu A, Nakamaru Y, Sugaya T, Kikuchi K, Yamada S, Wakisaka A, Kasai N, Koike T, Hatanaka M and Yoshiki T (1997) “A wide spectrum of collagen vascular and autoimmune diseases in transgenic rats carrying the env-pX gene of human T lymphocyte virus type I,” Int Immunol 9: 339-346). Microinjected ova are transferred into oviducts of pseudo pregnant rats. The integration of four transgenes in each offspring was confirmed using PCR, Southern blot, RT-PCR, northern blot and immunostaining. For establishment of each transgenic line, successful offspring for T- and M-tropic HIV-1 infection are maintained in each closed colony.

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Other embodiments are set forth within the following claims.

1. A rodent cell that replicates the HIV provirus and produces HIV particles.

2. The rodent cell of claim 1 wherein the rodent is a rat or a mouse.

3. The rodent animal cell of claim 2 producing HIV-1 virus particles.

4. A rodent animal cell stably expressing,
an active portion of a human CD4;
an active portion of a human chemokine receptor;
an active portion of a human CyclinT1; and
an active portion of a human Class II Transactivator.

5. The cell according to claim 4 wherein the human chemokine receptor is CXCR4 or CCR5.

6. The rodent animal cell of claim 5 wherein the HIV is HIV-1.

7. The cell according to claim 6 wherein the rodent is a rat or a mouse.

8. A method for preparing a rodent animal cell stably expressing an active portion of human CD4, an active portion of a human chemokine receptor, an active portion of a human CyclinT1, and an active portion of a human Class II Transactivator comprising,
introducing into a rodent animal cell a nucleotide coding for an active portion of a human CD4, a nucleotide coding for an active portion of a human chemokine receptor, a nucleotide coding for an active portion of a human CyclinT1, and a nucleotide coding for an active portion of a human CIITA;
incorporating each nucleotide into the genome of the rodent animal cell;
stably expressing in the rodent animal cell an active portion of human CD4, an active portion of a human chemokine receptor, an active portion of a human CyclinT1, and an active portion of a human Class II Transactivator.

9. The method of claim 8 wherein the cell is producing HIV virus particles.

10. The method of claim 9 wherein the HIV is HIV-1.

11. The method of claim 8 wherein the human chemokine receptor is CXCR4 or CCR5.

12. A transgenic rodent animal replicating HIV virus and producing HIV virus particles when infected with HIV.

13. The transgenic rodent animal of claim 12 wherein the HIV is HIV-1.

14. The transgenic rodent animal of claim 13, which is a rat or mouse.

15. The transgenic rodent animal of claim 13, wherein the animal is capable of developing HIV-1 disease.

16. The transgenic rodent animal of claim 13 exhibiting symptoms of HIV-1 infectious disease.

17. The transgenic rodent animal of claim 13 stably expressing a nucleotide coding for an active portion of human CD4, a nucleotide coding for an active portion of a human chemokine receptor, a nucleotide coding for an active portion of a human CyclinT1 and a nucleotide coding for an active portion of human Class II Transactivator.

18. The transgenic rodent animal of claim 17 wherein the human chemokine receptor is CXCR4 or CCR5.

19. A method for preparing a transgenic rodent animal capable of replicating HIV virus and producing HIV virus particles when infected with HIV, comprising:
introducing into an embryonic cell of a rodent animal a nucleotide coding for an active portion of human CD4, a nucleotide coding for an active portion of human chemokine receptor, a nucleotide coding for an active portion of human CyclinT1, and a nucleotide coding for an active portion of a human CIITA; and
developing the embryonic cell to obtain a transgenic rodent animal capable of replicating HIV virus and producing HIV virus particles when infected with HIV.

20. The method of claim 19 wherein the HIV is HIV-1.

21. The method of claim 20 wherein the nucleotide coding for an active portion of human CD4, the nucleotide coding for an active portion of a human chemokine receptor, the nucleotide coding for an active portion of human CyclinT1, and the nucleotide coding for an active portion of human CIITA are introduced into the embryonic cell on one or more plasmids.

22. The method of claim 21 wherein the one or more plasmids are selected from the group consisting of: pUC18 and pGEM-T Easy.

23. The method of claim 20 wherein the human chemokine receptor is one or more of CXCR4 or CCR5.

24. A method for assaying for anti-HIV-1 activity of a test agent, comprising,
contacting a transgenic rodent animal cell with a test agent, wherein the rodent animal cell stably expresses:
an active portion of human CD4;
an active portion of a human chemokine receptor;
an active portion of human CyclinT1; and
an active portion of human Class II Transactivator.
infecting the cell with HIV-1 virus; and
monitoring the level of HIV-1 RNA or a viral protein present in the cell.

25. The method of claim 24 wherein the human chemokine receptor is one or more of CXCR4 or CCR5.

26. A method of assaying for anti-HIV activity of a test agent, comprising,
providing an animal cell stably expressing
an active portion of human CD4;
an active portion of a human chemokine receptor;
an active portion of human CyclinT1; and
an active portion of human Class II Transactivator.
wherein the cell is infected with the HIV virus and is producing HIV virus particles;
contacting the animal cell with a test agent; and
monitoring the level of HIV RNA or a viral protein in the cell.

27. The method of claim 26 wherein the HIV is HIV-1.

28. The method of claim 26 wherein the cell is capable of producing HIV-1 virus particles.

29. The method of claim 26 wherein the human chemokine receptor is one or more of CXCR4 or CCR5.

30. A method for assaying an anti-HIV activity of a test agent, comprising:
administering a test agent to a transgenic rodent animal, which is infected with the HIV virus, is producing HIV virus, and is exhibiting symptoms of HIV infectious disease; and
monitoring the level in the blood of the animal one or more indices selected from the group consisting of: HIV RNA, circulating virus particles, CD4+ T-lymphocytes, viral proteins, and antibodies against viral proteins.

31. The method of claim 30 wherein the HIV is HIV-1.

32. A method for assaying an anti-HIV activity of a test agent, comprising the step of:
administering a test agent to a transgenic animal that replicates HIV virus and produces HIV virus particles when infected with HIV, and is stably expressing an active portion of human CD4, an active portion of human chemokine receptor, an active portion of human CyclinT1, and an active portion of human CIITA;
infecting the transgenic animal with HIV; and
monitoring the level of one or more indices selected from the group consisting of: HIV RNA, circulating virus particles, CD4+ T-lymphocytes, viral proteins and antibodies against viral proteins in said animal.

33. The method of claim 32 wherein the HIV is HIV-1.

34. The method of claim 33 wherein the human chemokine receptor is one or more of CXCR4 or CCR5.

35. A method for identifying an agent having an anti-HIV activity, comprising the steps of:
contacting a test agent with a transgenic animal cell that is stably expressing an active portion of human CD4; an active portion of a human chemokine receptor; an active portion of human CyclinT1; and an active portion of human Class II Transactivator;
wherein the cell is infected with the HIV virus and is producing HIV virus particles;
monitoring the level of HIV RNA or viral proteins in said cell; and
identifying an agent that inhibits HIV transcription or viral particle production as an agent having an anti-HIV activity.

36. The method of claim 35 wherein the HIV is HIV-1.

37. The method of claim 36 wherein the human chemokine receptor is one or more of CXCR4 or CCR5.

38. A method for identifying an agent having anti-HIV activity comprising:
contacting a test agent with a transgenic animal cell that is stably expressing an active portion of human CD4; an active portion of a human chemokine receptor; an active portion of human CyclinT1; and an active portion of human Class II Transactivator;
infecting the cell with HIV;
monitoring the level of HIV RNA or viral proteins in the cell; and
identifying an agent capable of inhibiting HIV transcription or viral particle production as an agent having an anti-HIV activity.

39. The method of claim 38 wherein the HIV is HIV-1.

40. The method of claim 39 wherein the human chemokine receptor is one or more of CXCR4 or CCR5.

41. A method for treating symptoms of HIV infection in a mammal, comprising:
administering a test agent to a transgenic rodent animal, which is producing HIV virus and is exhibiting symptoms of HIV infectious disease;
monitoring one or more symptoms associated with HIV infection in the animal; and
identifying an agent capable of alleviating one or more symptoms of HIV infection as an agent having an anti-HIV activity;
administering the agent having anti-HIV activity to the mammal.

42. The method of claim 41 wherein the HIV is HIV-1.

43. The method of claim 42 wherein the human chemokine receptor is one or more of CXCR4 or CCR5.

44. A method for treating and preventing symptoms of HIV infection in a mammal comprising:
administering a test agent to a transgenic animal that is replicates HIV-1 virus and produces HIV-1 virus particles when infected with HIV-1, and is stably expressing an active portion of human CD4; an active portion of a human chemokine receptor; an active portion of human CyclinT1; and an active portion of human Class II Transactivator;
introducing HIV virus into the animal;
monitoring one or more symptoms associated with HIV infection in the animal;
identifying an agent that alleviates or prevents one or more symptoms of HIV infection; and
treating the mammal with the agent identified.

45. The method of claim 44 wherein the HIV is HIV-1.

46. The method of claim 45 wherein the human chemokine receptor is one or more of CXCR4 or CCR5.

47. The agent identified by the method of claim 35.

48. The agent identified by the method of claim 38.

49. A pharmaceutical composition for preventing or treating HIV-1 infectious disease comprising the agent identified by the method of claim 35.

50. A pharmaceutical composition for preventing or treating HIV-1 infectious disease comprising the agent identified by the method of claim 37.

51. A pharmaceutical composition for preventing or treating HIV-1 infectious disease comprising the agent identified by the method of claim 38.

52. A pharmaceutical composition for preventing or treating HIV-1 infectious disease comprising the agent identified by the method of claim 40.

53. The use of the cell of claim 3 for identifying an agent having an anti-HIV activity.

54. The use of claim 53 wherein the HIV is HIV-1.

55. The use of the cell of claim 6 for identifying an agent having an anti-HIV activity.

56. The use of claim 55 wherein the HIV is HIV-1.

57. The use of the animal according to claim 12 for identifying an agent having an anti-HIV activity.

58. The use of claim 57 wherein the HIV is HIV-1.

59. The use of the animal according to claim 18 for identifying an agent having an anti-HIV activity.

60. The use of claim 59 wherein the HIV is HIV-1.
